(19) United States
(12) Patent Application Publication
(10) Pub. No.: US 2018/0311238 A1
(43) Pub. Date: Nov. 1, 2018

(54) Selective Agonist Of a6 Containing nAChRs

(71) Applicant: Saniona A/S, Ballerup (DK)

(72) Inventors: Tino Dyhring, Ballerup (DK); Dan Peters, Ballerup (DK); Palle Christophersen, Ballerup (DK); Karin Sandager Nielsen, Ballerup (DK); Lars Ulrik Wahlberg, Tiverton, RI (US); Dipak Amrutkar, Ballerup (DK)

(21) Appl. No.: 15/964,771

(22) Filed: Apr. 27, 2018

(30) Foreign Application Priority Data
Apr. 28, 2017 (EP) 17168636.3

(51) Int. Cl. A61K 31/4995

(52) U.S. Cl. CPC A61K 31/4995; A61K 31/16; A61K 2300/00

(57) ABSTRACT

The present invention relates to a subtype selective partial agonist of α6 containing nicotinic acetylcholine receptors. Due to its uniquely selective and functional profile, 9-methyl-3-pyridin-3-yl-3,9-diaza-bicyclo[3.3.1]nonane may be useful in the treatment, prevention and/or alleviation of a disease, disorder and/or condition which is responsive to activation of a nicotinic acetylcholine receptor (nAChR) in a subject, wherein the nAChR comprises at least one cholinergic receptor nicotinic alpha 6 subunit (nAChRa6). Preferably, said disease, disorder and/or condition is a Parkinsonian disorder or pain.

### CROSS REFERENCE TO RELATED APPLICATION

This application claims the benefit of European Patent Application No. 17168636.3, filed Apr. 28, 2017, the entirety of which is incorporated by reference herein.

### TECHNICAL FIELD

The present invention relates to a subtype selective partial agonist of α₆ containing nicotinic acetylcholine receptors. Due to its uniquely selective and functional profile, 9-methyl-3-pyridin-3-yl-3,9-diaza-bicyclo[3.3.1]nonane may be useful in the treatment, prevention and/or alleviation of a disease, disorder and/or condition which is responsive to activation of a nicotinic acetylcholine receptor (nAChR) in a subject, wherein the nAChR comprises at least one cholinergic receptor nicotinic alpha 6 subunit (nAChRα₆). Preferably, said disease, disorder and/or condition is a Parkinsonian disorder or pain.

### BACKGROUND

The symptoms associated with Parkinson's disease are the result of malfunctioning neurotransmitter systems in the brain, most notably dopamine (DA). Symptoms worsen over time as more and more of the cells affected by the disease are lost. Degeneration of DA neurons is particularly evident in the substantia nigra pars compacta (SNc), which projects to the dorsolateral striatum. The loss of striatal DA increases the excitatory drive in the basal ganglia, disrupting voluntary motor control and causing the characteristic motor deficits of Parkinson's disease. However, other neurotransmitter systems in the striatum also play a significant role for motor control, including the nicotinic cholinergic system. Indeed, there is an extensive anatomical overlap between the dopaminergic and cholinergic systems, and acetylcholine is well known to modulate striatal DA release both in vitro and in vivo [1-4]. Accumulating evidence suggests that nicotinic acetylcholine receptor (nAChR) modulation of dopaminergic function may be of benefit in neurological disorders such as Parkinson's disease. Hence, it has been demonstrated that activation of nAChRs can have a neuroprotective effect and nicotine has been shown to protect against nigrostriatal damage through an interaction with nAChRs in several parkinsonian animal models [3, 5-7], findings that may explain the well-established decline in disease incidence with tobacco use [8]. In addition, nicotine improves levodopa-induced abnormal involuntary movements, a debilitating complication of dopamine replacement therapy [3, 7]. These combined observations suggest that nAChR stimulation represents a useful treatment strategy for neuroprotection and symptomatic treatment in Parkinson's disease. However, nicotine itself is poorly suited for use as a therapeutic drug due to its many adverse events caused by the non-selective action on all nAChRs subtypes in the brain and in the periphery.

Nicotinic acetylcholine receptors are ion channels composed of five subunits, with the predominant subtypes in the brain being α₄β₂* (the asterisk indicates the possible presence of other subunits in the receptor complex) and α₇ receptors, whereas the peripheral autonomous ganglionic and neuromuscular receptors are composed of α₃β₄ and α₁β₁δε, respectively. In DA neurons and its striatal projections, α₄β₂* and α₆β₂* receptors dominate. The modulatory control of dopaminergic function exerted by the α₄β₂* and α₆β₂* nAChR subtypes may play a pivotal role in the functional changes observed with nigrostriatal dopamine degeneration. Support for the involvement of nigrostriatal α₆ containing nAChRs in relation to motor control comes from parkinsonian animal models in which the nigrostriatal pathway is selectively damaged with dopaminergic neurotoxins such as 6-hydroxydopamine (6-OHDA) or 1-methyl-4-phenyl-1,2,3,6-tetrahydropyridine (MPTP). Such lesions result in a decrease in α6 containing nAChR expression and function that closely parallels the decline in dopaminergic terminal integrity [9]. Results in parkinsonian animal models therefore seem to parallel those in post mortem Parkinson's disease brains, where large declines in α6-containing nAChRs in the striatum are observed, which also correlate with the magnitude of the DA transporter loss (a marker for functioning DA neurons) [10, 11]. The nicotinic α6 subunit is also known to be localized in sensory ganglia [12-15]. These constitute neurons that convert a specific type of stimulus into action potential through a process called sensory transduction. This sensory information travels along afferent nerve fibres in an afferent or sensory nerve, to the brain via the spinal cord and is also involved in nociception, which usually causes the perception of pain. Nicotine itself has been demonstrated to exert anti-allodynic effects after both inflammatory and neuropathic injuries [16]. Data suggest that nicotine blocks mechanical allodynia in the periphery and/or spinal cord in a wholly α6-specific manner, except supraspinally, where both α₆* and α₄* nicotinic receptors appear to contribute [17]. Hence, α₆-containing nAChRs may represent unique targets for the treatment of neurodegenerative disorders characterized by nigrostriatal damage, such as Parkinson's disease as well as chronic pain.

### SUMMARY OF INVENTION

9-Methyl-3-pyridin-3-yl-3,9-diaza-bicyclo[3.3.1]nonane is a subtype selective partial agonist of α₆ containing receptors with basically no functional agonist activity on other nicotinic receptors. Importantly, the present inventors have demonstrated that 9-methyl-3-pyridin-3-yl-3,9-diaza-bicyclo[3.3.1]nonane has basically no agonist activity on α7- and α1-containing receptors, which are associated with many adverse events upon activation. Furthermore, 9-methyl-3-pyridin-3-yl-3,9-diaza-bicyclo[3.3.1]nonane stimulates dopamine release, and is neuroprotective for dopaminergic neurons. The present inventors have also demonstrated that 9-methyl-3-pyridin-3-yl-3,9-diaza-bicyclo[3.3.1]nonane may be used to treat tremors associated with dopamine dysfunctionas well as to alleviate L-dopa-induced dyskinesia.

Due to its uniquely selective and functional profile, 9-methyl-3-pyridin-3-yl-3,9-diaza-bicyclo[3.3.1]nonane is a potential drug candidate for treatment of Parkinson's disease and chronic pain patients.

In one aspect, the current invention concerns a method for treatment, prevention and/or alleviation of a disease, disorder and/or condition which is responsive to activation of a nicotinic acetylcholine receptor (nAChR) in a subject, wherein the nAChR comprises at least one cholinergic receptor nicotinic alpha 6 subunit (nAChRα₆), the method comprising administering a therapeutically effective amount of 9-methyl-3-pyridin-3-yl-3,9-diaza-bicyclo[3.3.1]nonane to said subject in need thereof.

In one aspect, the current invention concerns a pharmaceutical composition comprising 9-methyl-3-pyridin-3-yl-3,9-diaza-bicyclo[3.3.1]nonane, or a pharmaceutically acceptable salt thereof, and L-DOPA.

In another aspect, the current invention concerns a kit of parts comprising 9-methyl-3-pyridin-3-yl-3,9-diaza-bicyclo[3.3.1]nonane, or a pharmaceutically acceptable salt thereof, and L-DOPA, for simultaneous, successive or separate administration.

In a further aspect, the current invention concerns a method of activating a nAChR in a subject, wherein the nAChR comprises at least one nAChRα₆, the method comprising administering 9-methyl-3-pyridin-3-yl-3,9-diaza-bicyclo[3.3.1]nonane or a pharmaceutically acceptable salt thereof.

9-Methyl-3-pyridin-3-yl-3,9-diaza-bicyclo[3.3.1]nonane is able to stimulate dopamine (DA) release from isolated striatal DA terminals, it passes the blood brain barrier, and it is able to displace selective radioactive ligands from nicotinic receptors demonstrating robust target engagement in vivo. Hence, in one aspect, the current invention concerns a method for inducing dopamine release from a neuron expressing a nAChR, wherein the nAChR comprises at least one nAChRα6, the method comprising administering a therapeutically effective amount of 9-methyl-3-pyridin-3-yl-3,9-diaza-bicyclo[3.3.1]nonane to said neuron.

In one aspect, the current invention concerns a method for stimulating neuronal survival of a neuron expressing a nAChR, wherein the nAChR comprises at least one nAChRα₆, the method comprising administering a therapeutically effective amount of 9-methyl-3-pyridin-3-yl-3,9-diaza-bicyclo[3.3.1]nonane to said neuron.

In yet another aspect, the current invention concerns a method for diagnosis of a disease, disorder and/or condition which is responsive to activation of a nAChR in a subject, wherein the nAChR comprises at least one nAChRα₆, the method comprising the steps of:
a) Administering labelled 9-methyl-3-pyridin-3-yl-3,9-diaza-bicyclo[3.3.1]nonane to a subject;
b) Detecting the signals from the labelling moiety in a).

### DESCRIPTION OF DRAWINGS

FIG. 1. Characterization of 9-methyl-3-pyridin-3-yl-3,9-diaza-bicyclo[3.3.1]nonane at selected nAChRs measured as intracellular calcium changes in fluorescence-based assays. Stimulated changes in the intracellular calcium concentration are measured at various concentrations of 9-methyl-3-pyridin-3-yl-3,9-diaza-bicyclo[3.3.1]nonane, and peak fluorescent responses at the individual test concentrations are expressed as a percentage of a control response (maximal effective concentration of nicotine). Concentration-response curves are plotted for each of the nAChRs tested in order to determine EC₅₀ and efficacy values for 9-methyl-3-pyridin-3-yl-3,9-diaza-bicyclo[3.3.1]nonane.

FIG. 2. Characterization of 9-methyl-3-pyridin-3-yl-3,9-diaza-bicyclo[3.3.1]nonane at selected nAChRs measured as compound-evoked currents in oocytes. Evoked currents are at various concentrations of 9-methyl-3-pyridin-3-yl-3,9-diaza-bicyclo[3.3.1]nonane, and peak currents obtained at the individual test concentrations are expressed as a percentage of a control response (maximal effective concentration of acetylcholine). Concentration-response curves are plotted for each of the nAChRs tested in order to determine EC₅₀ and efficacy values for 9-methyl-3-pyridin-3-yl-3,9-diaza-bicyclo[3.3.1]nonane.

FIG. 3. In vivo time course study for brain exposure of 9-methyl-3-pyridin-3-yl-3,9-diaza-bicyclo[3.3.1]nonane determined by displacement of ³H-epibatidine. Mice were dosed orally with 9-methyl-3-pyridin-3-yl-3,9-diaza-bicyclo[3.3.1]nonane (2 mg/kg) and the specific binding of ³H-epibatidine was determined at time points up to 6 hours. Exposure of 9-methyl-3-pyridin-3-yl-3,9-diaza-bicyclo[3.3.1]nonane is depicted as percentage inhibition of the specific binding of ³H-epibatidine.

FIG. 4. Characterization of 9-methyl-3-pyridin-3-yl-3,9-diaza-bicyclo[3.3.1]nonane-mediated ³H-dopamine release from rat striatal synaptosomes. Purified nerve terminals isolated from rat striatum are stimulated with various concentrations of 9-methyl-3-pyridin-3-yl-3,9-diaza-bicyclo[3.3.1]nonane, and release of 3H-dopamine is depicted as the fraction of release relative to the total amount of releasable dopamine (FR %). Release induced by a potassium-mediated depolarization (30 mM KCl) is depicted for comparison.

FIG. 5A. Depicts power spectra of male Sprague Dawley rats assessed in automated tremor monitors (San Diego Instruments, Tremor Monitor™), showing the effects of the individual treatments on the full power spectra evaluated for 30 min. Shaded areas depict the three different frequency ranges selected for calculation of the AUCs.

FIG. 5B. Depicts power spectra of male Sprague Dawley rats assessed in automated tremor monitors with AUC of the frequency range 3-13 Hz. Vehicle+vehicle treated animals were tested as n=4. All other groups were tested as n=8.

FIG. 5C. Depicts power spectra of male Sprague Dawley rats assessed in automated tremor monitors with AUC of the frequency range 20-35 Hz. Vehicle+vehicle treated animals were tested as n=4. All other groups were tested as n=8.

FIG. 5D. Depicts power spectra of male Sprague Dawley rats assessed in automated tremor monitors with AUC of the frequency range 40-63 Hz, respectively. Vehicle+vehicle treated animals were tested as n=4. All other groups were tested as n=8.

FIG. 6. Effect of 9-methyl-3-pyridin-3-yl-3,9-diaza-bicyclo[3.3.1]nonane (Cmpd) on primary dopaminergic neuronal culture injured by MPP+ (4 uM, 48H) expressed in percentage of control. Data is expressed as mean±SEM (6 data points per condition). A global analysis of the data was performed using a one-way analysis of variance (ANOVA) followed by Dunnett's test. The level of significance is set at p<0.05. # represents the condition of intoxication; * p<0.05; ** p<0.01; and *** p<0.001.

FIG. 7A. Depicts effects of 9-methyl-3-pyridin-3-yl-3,9-diaza-bicyclo[3.3.1]nonane on dyskinesia in Parkinsonian rats. Administration of 9-methyl-3-pyridin-3-yl-3,9-diaza-bicyclo[3.3.1]nonane produced a significant, dose-related decrease in AIMs with the 0.3 and 1.0 mg/kg doses reaching statistical significance relative to saline controls.

FIG. 7B. Depicts effects of 9-methyl-3-pyridin-3-yl-3,9-diaza-bicyclo[3.3.1]nonane on dyskinesia in Parkinsonian rats. Treatment with a follow-up dose of 3.0 mg/kg of 9-methyl-3-pyridin-3-yl-3,9-diaza-bicyclo[3.3.1]nonane did not further decrease L-dopa-induced AIMs.

### DETAILED DESCRIPTION

The present invention relates to administration of 9-methyl-3-pyridin-3-yl-3,9-diaza-bicyclo[3.3.1]nonane (depicted below).

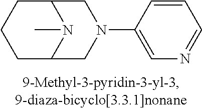 [SMILES: CN1C2CCCC1CN(c1cccnc1)C2]

Methods of Preparation

9-Methyl-3-pyridin-3-yl-3,9-diaza-bicyclo[3.3.1]nonane fumaric acid salt may be prepared as described in WO 2007/090888. Other salts may be prepared by methods known by those of skill in the art.

Biological Activity

The present invention concerns 9-methyl-3-pyridin-3-yl-3,9-diaza-bicyclo[3.3.1]nonane as a ligand and modulator of cholinergic receptor nicotinic alpha 6 subunit (nAChRα₆).

Method of Treatment

In one aspect, the current invention concerns a method for treatment, prevention and/or alleviation of a disease, disorder and/or condition which is responsive to activation of a nicotinic acetylcholine receptor (nAChR) in a subject, wherein the nAChR comprises at least one nAChRα₆, the method comprising administering a therapeutically effective amount of 9-methyl-3-pyridin-3-yl-3,9-diaza-bicyclo[3.3.1]nonane to said subject in need thereof. In one embodiment, said method concerns preventing said disease, disorder and/or condition. In one embodiment, said method concerns alleviating said disease, disorder and/or condition. In a preferred embodiment, said method concerns treating said disease, disorder and/or condition.

In one embodiment, the present invention relates to a method for treating a Parkinsonian disorder, pain, and/or a systemic atrophy primarily affecting the central nervous system in a subject, the method comprising administering a therapeutically effective amount of 9-methyl-3-pyridin-3-yl-3,9-diaza-bicyclo[3.3.1]nonane, or a pharmaceutically acceptable salt thereof, to said subject in need thereof.

In one aspect, the current invention concerns a kit of parts comprising 9-methyl-3-pyridin-3-yl-3,9-diaza-bicyclo[3.3.1]nonane, or a pharmaceutically acceptable salt thereof, and L-DOPA, for simultaneous, successive or separate administration. Said kit can be used for treating a disease, disorder and/or condition which is responsive to activation of a nicotinic acetylcholine receptor (nAChR) in a subject, wherein the nAChR comprises at least one cholinergic receptor nicotinic alpha 6 subunit (nAChRα₆). In one embodiment, said kit is used for treating a Parkinsonian disorder, pain, and/or a systemic atrophy primarily affecting the central nervous system. In particular, said kit can be used for treating Levodopa-induced dyskinesia (LID). In one embodiment, said kit further comprises Benserazide or Carbidopa.

Preferably, 9-methyl-3-pyridin-3-yl-3,9-diaza-bicyclo[3.3.1]nonane act as an agonist on nAChRα₆.

9-Methyl-3-pyridin-3-yl-3,9-diaza-bicyclo[3.3.1]nonane is considered useful for the for the treatment, prevention and/or alleviation of a disease, disorder and/or condition which is responsive to activation of a nAChR in a subject, wherein the nAChR comprises at least one nAChRα₆.

In some embodiments, 9-methyl-3-pyridin-3-yl-3,9-diaza-bicyclo[3.3.1]nonane is useful for the treatment, prevention or alleviation of a disease of the nervous system. In one embodiment, said disease of the nervous system is a systemic atrophy primarily affecting the central nervous system, such as diseases and disorders classified in G10-G14 of the World Health Organization's 10th revision of the International Statistical Classification of Diseases and Related Health Problems (ICD-10). Preferably, said systemic atrophy primarily affecting the central nervous system is Huntington's disease or ataxia (such as spinocerebellar atrophies (SCA)). In one embodiment, said disease of the nervous system is an extrapyramidal disorder or a movement disorder. Said extrapyramidal disorder or movement disorder preferably includes disorders classified in G20-G26 of the World Health Organization's 10th revision of the International Statistical Classification of Diseases and Related Health Problems (ICD-10).

Preferably, the extrapyramidal disorder and/or movement disorder is selected from the group consisting of Parkinson's disease, parkinsonism, and dystonia.

In one embodiment, said disease, disorder and/or condition is a Parkinsonian disorder. The Parkinsonian disorder may be selected from the group consisting of Parkinson disease (PD), corticobasal degeneration (CBD), progressive supranuclear palsy (PSP), multiple system atrophy (MSA), dementia with Lewy bodies (DLB), Parkinson disease dementia, Levodopa-induced dyskinesia (LID), spinocerebellar atrophies (SCA), and frontotemporal dementia (FTD). Preferably, said disease, disorder and/or condition is LID.

Parkinsonism is a clinical syndrome characterized by lesions in the basal ganglia, predominantly in the substantia nigra. Preferably, said Parkinsonian disorder is Parkinson's disease (PD) or other diseases affecting the basal ganglia/striatal system.

In one embodiment, 9-methyl-3-pyridin-3-yl-3,9-diaza-bicyclo[3.3.1]nonane is useful in the treatment, prevention or alleviation of dyskinesia resulting from long-term dopamine therapy, such as long-term treatment with L-DOPA. In Example 8, the present inventors have demonstrated that 9-methyl-3-pyridin-3-yl-3,9-diaza-bicyclo[3.3.1]nonane is able to alleviate L-dopa-induced dyskinesia.

In another embodiment, the disease, disorder and/or condition is pain, mild or moderate or even severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

Activation of nAChR

In another aspect, the current invention concerns a method of activating a nAChR in a subject, wherein the nAChR comprises at least one nAChRα6, the method comprising administering 9-methyl-3-pyridin-3-yl-3,9-diaza-bicyclo[3.3.1]nonane or a pharmaceutically acceptable salt thereof. Preferably, 9-methyl-3-pyridin-3-yl-3,9-diaza-bicyclo[3.3.1]nonane acts as an agonist on said nAChRα6.

Induction of Dopamine Release

Administering 9-methyl-3-pyridin-3-yl-3,9-diaza-bicyclo[3.3.1]nonane to a neuron expressing a nAChR, wherein the nAChR comprises at least one nAChRα₆, may induce dopamine release. Preferably, said neuron is a neuron in substantia nigra pars compacta. In other embodiments the neuron is a neuron of the sensory ganglia.

Neuronal Survival

In one aspect, the current invention concerns a method for stimulating neuronal survival of a neuron expressing a nAChR, wherein the nAChR comprises at least one nAChRα₆, the method comprising administering a therapeutically effective amount of 9-methyl-3-pyridin-3-yl-3,9-diaza-bicyclo[3.3.1]nonane to said neuron.

In one embodiment, said neuron is a dopaminergic neuron. In one embodiment, said neuron is a tyrosine hydroxylase (TH)-positive neuron.

Diagnostic Methods

9-Methyl-3-pyridin-3-yl-3,9-diaza-bicyclo[3.3.1]nonane may also be useful as a diagnostic tool or monitoring agent in various diagnostic methods, and in particular for in vivo receptor imaging (neuroimaging), and it may be used in labelled or unlabelled form. Hence, in one aspect, the current invention concerns a method for diagnosis of a disease, disorder and/or condition which is responsive to activation of a nAChR in a subject, wherein the nAChR comprises at least one nAChRα₆, the method comprising the steps of:
a) Administering labelled 9-methyl-3-pyridin-3-yl-3,9-diaza-bicyclo[3.3.1]nonane to a subject;
b) Detecting the signals from the labelling moiety in a).

The labelling of 9-methyl-3-pyridin-3-yl-3,9-diaza-bicyclo[3.3.1]nonane may be made by conjugation of a detectable moiety, such as a radioactive atom, such as ¹¹C or ¹⁸F, or group. The labelling of 9-methyl-3-pyridin-3-yl-3,9-diaza-bicyclo[3.3.1]nonane may also be made by exchange of one or more atoms to the corresponding radioactive isotope, such as ¹¹C.

Preferably, the detection is made by position emission tomography (PET).

In one embodiment, the method is used for estimation of number of neurons in substantia nigra pars compacta. The method may also be used for monitoring the development of the disease, disorder and/or condition. Preferably, said disease, disorder and/or condition is a disease of the nervous system, which may be a systemic atrophies primarily affecting the central nervous system, a extrapyramidal disorder or a movement disorder. Preferably, said systemic atrophy primarily affecting the central nervous system is Huntington's disease or ataxia (such as spinocerebellar atrophies (SCA)). Preferably, the extrapyramidal disorder and/or movement disorder is selected from the group consisting of Parkinson's disease, parkinsonism, and dystonia.

Pharmaceutically Acceptable Salts

9-Methyl-3-pyridin-3-yl-3,9-diaza-bicyclo[3.3.1]nonane may be provided in any form suitable for the intended administration. Suitable forms include pharmaceutically (i.e. physiologically) acceptable salts.

Examples of pharmaceutically acceptable addition salts include, without limitation, the non-toxic inorganic and organic acid addition salts such as the fumarate derived from fumaric acid, the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Preferably, 9-methyl-3-pyridin-3-yl-3,9-diaza-bicyclo[3.3.1]nonane is administered as a fumaric acid salt.

Other acids such as oxalic acid, which may not be considered pharmaceutically acceptable, may be useful in the preparation of salts useful as intermediates in obtaining a chemical compound of the invention and its pharmaceutically acceptable acid addition salt.

Additional examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of 9-methyl-3-pyridin-3-yl-3,9-diaza-bicyclo[3.3.1]nonane include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of 9-Methyl-3-pyridin-3-yl-3,9-diaza-bicyclo[3.3.1]nonane include compounds modified at one or more reactive or derivatizable groups of the parent compound. Of particular interest are compounds modified at a carboxyl group, a hydroxyl group, or an amino group. Examples of suitable derivatives are esters or amides.

9-Methyl-3-pyridin-3-yl-3,9-diaza-bicyclo[3.3.1]nonane may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Pharmaceutical Compositions

While 9-methyl-3-pyridin-3-yl-3,9-diaza-bicyclo[3.3.1]nonane may be administered in the form of the raw chemical compound, it is preferred to introduce a therapeutically effective amount of the active ingredient, optionally in the form of a physiologically acceptable salt, in a pharmaceutical composition together with one or more pharmaceutically acceptable adjuvant, excipient, carrier, buffer, diluent, and/or other customary pharmaceutical auxiliary. The term “acceptable” is used herein in the sense of being compatible with the other ingredients of the formulation and not harmful to the recipient thereof.

In one embodiment, the invention provides pharmaceutical compositions comprising 9-methyl-3-pyridin-3-yl-3,9-diaza-bicyclo[3.3.1]nonane, or a pharmaceutically acceptable salt thereof, together with one or more pharmaceutically acceptable carriers, and, optionally, other therapeutic and/or prophylactic ingredients, known and used in the art.

In one aspect, the current invention concerns a composition comprising 9-methyl-3-pyridin-3-yl-3,9-diaza-bicyclo[3.3.1]nonane, or a pharmaceutically acceptable salt thereof, and L-DOPA, i.e. said other therapeutic is L-DOPA. Today, L-DOPA is used to increase dopamine concentrations in the treatment of e.g. Parkinson's disease and dopamine-responsive dystonia. In one embodiment, said pharmaceutical composition further comprises a compound capable of preventing break-down of 9-methyl-3-pyridin-3-yl-3,9-diaza-bicyclo[3.3.1]nonane and/or L-DOPA. Thus, in one embodiment, said composition comprises Benserazide and/or Carbidopa.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use. Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Route of Administration

The pharmaceutical composition of the invention may be administered by any convenient route, which suits the desired therapy. Preferred routes of administration include oral administration, in particular in tablet, in capsule, in drag& in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

Dosage

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of the active pharmaceutical ingredient (API) per individual dose, preferably of from about 1 to about 100 mg, most preferred of from about 1 to about 10 mg, are suitable for therapeutic treatments. The dosage is calculated from 9-methyl-3-pyridin-3-yl-3,9-diaza-bicyclo[3.3.1]nonane free base.

The active ingredient may be administered in one or several doses per day. A satisfactory result can, in certain instances, be obtained at a dosage as low as 5 μg/kg.

The upper limit of the dosage range is presently considered to be about 10 mg/kg. Preferred ranges are from about 5 μg/kg to about 10 mg/kg/day, such as about from 50 μg/kg to 5 mg/kg/day, such as about from 100 μg/kg to 1 mg/kg/day.

It is at present contemplated that a suitable dosage of the active pharmaceutical ingredient (API) is 0.1-500 mg API per day, for example 1-100 mg API per day, such as 5-50 mg API per day, such as 10-30 mg API per day. However, the dosage is dependent upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Characterization of 9-methyl-3-pyridin-3-yl-3,9-diaza-bicyclo[3.3.1]nonane at nAChRs Measured in FLIPR

The level of agonist activity of 9-methyl-3-pyridin-3-yl-3,9-diaza-bicyclo[3.3.1]nonane was tested in functional fluorescence-based calcium assays using TE671 cells and HEK293 cells stably expressing human α₆/α₃β₂β₃^{V273S}, α₃β₄ and α₄β₂ nicotinic receptors.

FLIPR Assays

Cells were plated on poly-D-lysine coated 384-well microtiter plates and were allowed to proliferate for 24 h. Dye loading was performed by incubating cells with 2 μM fluo-4/AM for 1.5 h at room temperature. Dye not taken up by cells was removed by aspiration followed by three washing cycles with 25 μl of NMDG Ringer buffer (in mM: 140 NMDG, 5 KCl, 1 MgCl₂, 10 CaCl₂, 10 HEPES, pH 7.4) after which the cells were kept in 25 μl of the same buffer. The microtiter plates were placed in a Fluoremetric Imaging Plate Reader (FLIPR) and subjected to test compound at various concentrations. Background subtracted compound-mediated calcium responses were normalized to 100 μM nicotine control responses and pEC₅₀ as well as relative maximal efficacy values were determined.

Results

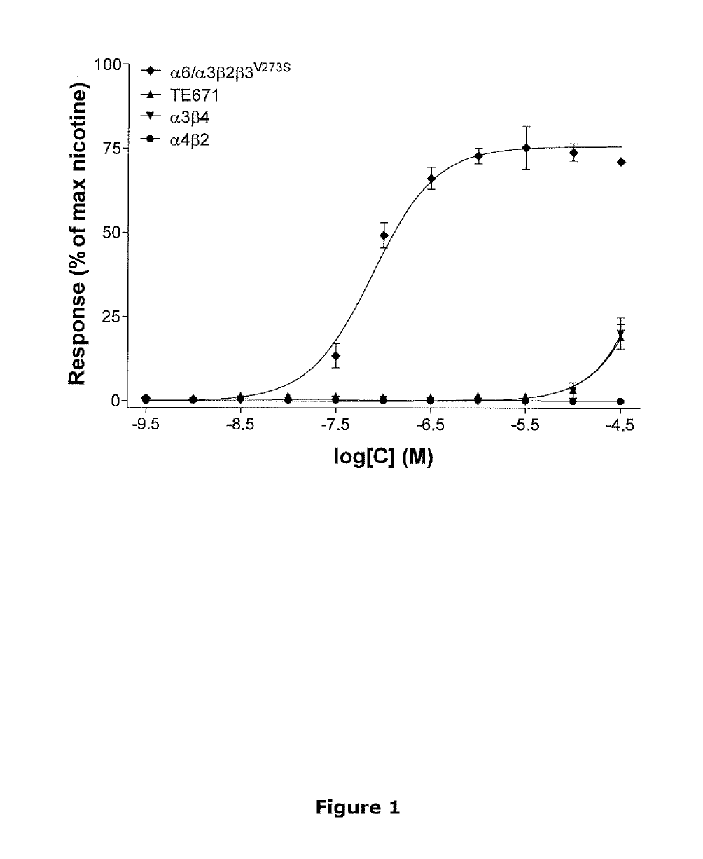

The data (see FIG. 1) demonstrate a high level of selectivity for α₆-containing nAChRs, with practically no efficacy at α₃β₄, α₄β₂ and the neuromuscular α₁-containing receptor subtypes. 9-Methyl-3-pyridin-3-yl-3,9-diaza-bicyclo[3.3.1]nonane displayed an EC₅₀ value of 71 nM when tested at α₆/α₃β₂β₃^{V273S}.

### Example 2

### Characterization of 9-methyl-3-pyridin-3-yl-3,9-diaza-bicyclo[3.3.1]nonane at nAChRs Measured on Oocytes

Oocyte Electrophysiology Assays

Two-electrode voltage-clamp electrophysiology recordings were done in Xenopus laevis oocytes injected with approximately 25 ng cRNA. After injection, oocytes were incubated at 17° C. for 2-3 days. During measurements, an oocyte was placed in a custom designed recording chamber where compound solutions are added directly to the oocyte via a glass capillary. Compound solutions were prepared on the day of measurement and applied to oocytes with a flowrate of 2.0 ml/min. All datasets were baseline subtracted and responses to individual applications were read as peak current amplitudes. Concentration response relationships describing compound effect at a fixed acetylcholine concentration were fitted to a monophasic Hill-equation. Potency (EC₅₀) and efficacy values (fitted maximal current relative to maximal current of acetylcholine).

Results

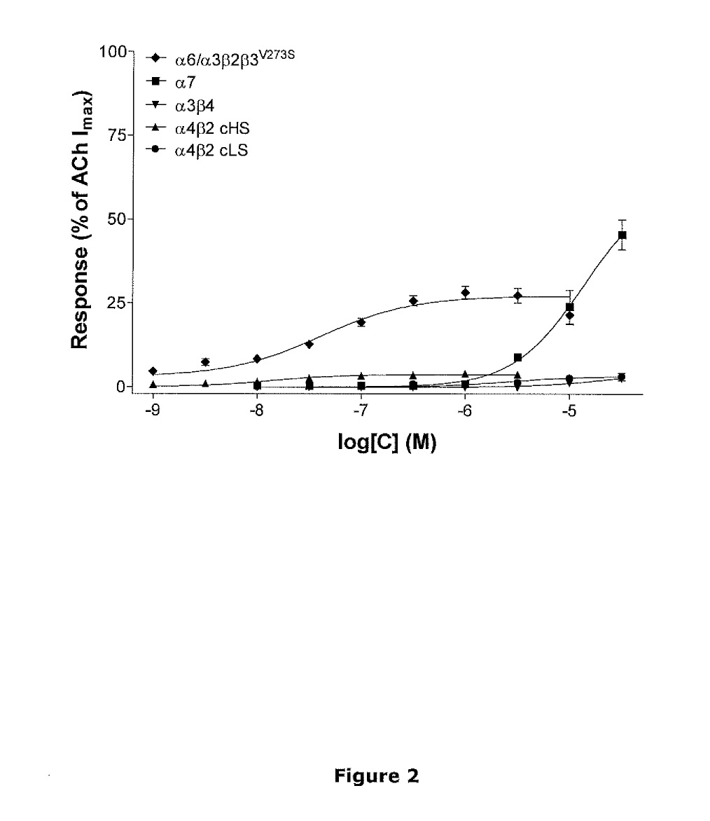

9-Methyl-3-pyridin-3-yl-3,9-diaza-bicyclo[3.3.1]nonane exhibited an EC₅₀ value of 52 nM when tested at the α₆/α₃β₂β₃^{V273S} receptor and with a maximal efficacy of 27% compared to ACh (see FIG. 2). An EC₅₀ value of 13 μM was attained at α7, whereas practically no efficacy was observed at α₃≈₄ and α₄β₂ cHS/cLS receptors. Hence, 9-methyl-3-pyridin-3-yl-3,9-diaza-bicyclo[3.3.1]nonane display a marked functional selectivity for α₆-containing receptors, whereas basically no functional selectivity is displayed for α₇-, α₃β₄- or α₄β₂-containing receptors.

### Example 3

### Determination of 9-methyl-3-pyridin-3-yl-3,9-diaza-bicyclo[3.3.1]nonane Binding Affinity to Nicotinic Receptors

In Vitro Inhibition of ³H-epibatidine Binding to HEK Cells Expressing the Human Nicotinic α₆α₃/β₂/β₃^{V273S} Receptor

Epibatidine is an alkaloid that was first isolated from the skin of the Ecuadorian frog Epipedobates tricolor and was found to have very high affinity for neuronal nAChRs, where it acts as a potent agonist. The high affinity binding site for ³H-epibatidine is most certainly binding to the α₄β₂ subtype of nicotinic receptors. However, ³H-epibatidine can also be used for receptor binding studies to human α₆-containing receptors expressed in mammalian cells.

Tissue Preparation

HEK293 cells with stable expression of recombinant human nicotinic α₆α₃/β₂/β₃^{V273S} receptors were seeded in T175 polystyrene flasks and cultured (37° C., 5% CO₂) in Dulbecco's Modified Eagle Medium (DMEM) with GlutaMAX™ supplemented with 10% foetal bovine serum and the antibiotics Hygromycin B (0.15 mg/ml; α₆α₃ subunit) and G418 (0.5 mg/ml; β₃^{V273S} subunit). When the cultures reached confluency, the DMEM was removed and cells were rinsed once with 10 ml of Dulbecco's Phosphate Buffered Saline (DPBS). Following addition of 10 ml DPBS to the cultures for approximately 5 min, cells were easily detached from the surface by shaking or tapping the flask gently. The cell suspension was transferred to Falcon tubes, and the culture flask was rinsed once with DPBS. The combined cell suspensions were centrifuged at 23,500×g for 10 min at 2° C. The pellet was washed once in 10 ml Tris, HCl buffer (50 mM, pH 7.4) using an Ultra-Turrax homogenizer and centrifuged at 2° C. for 10 min at 27,000×g. The washed pellet was re-suspended in 10 ml Tris, HCl buffer and frozen at −80° C. until the day of the binding experiment.

Assay

On the day of the experiment, cells were thawed and centrifuged for 10 min (27,000×g) at 2° C. The pellet was re-suspended in ice-cold Tris, HCl buffer (50 mM, pH 7.4) using an Ultra-Turrax homogenizer to 50-100 μg protein per assay and used for binding assays (typically tissue from one T175 flask in 500 ml buffer). Aliquots of 8.0 ml cell suspension were added to 200 μl of test compound solution and 200 μl of ³H-epibatidine (0.03 nM, final concentration), mixed and incubated for 4 h at 25° C. Non-specific binding was determined using 30 μM (−)-nicotine.

Solutions of test compounds and ³H-epibatidine were prepared 42× the desired final concentration. Compounds were dissolved in 100% DMSO (10 mM stock), diluted in 48% ethanol-water, and tested in triplicate in serial 1:3 dilutions.

Binding was terminated by rapid filtration onto Whatman GF/C glass fibre filters (pre-soaked in 0.1% polyethyleneimine for at least 30 min). Filters were immediately washed with 2×5 ml of ice-cold Tris, HCl buffer.

The amount of radioactivity on the filters was determined by conventional liquid scintillation counting using a Tri-Carb™ counter (PerkinElmer Life and Analytical Sciences). Specific binding was calculated as total binding minus non-specific binding.

In Vitro Inhibition of ³H-Cytisine Binding

The predominant subtype with high affinity for nicotine is comprised of α₄ and β₂ subunits. Here, the nicotine agonist ³H-cytisine is used to selectively label nAChRs of the α₄β₂ subtype.

Tissue Preparation

Preparations were performed at 0-4° C. Cerebral cortices from male Wistar rats (150-250 g) were homogenized for 20 sec in 15 ml Tris-HCl (50 mM, pH 7.4) containing 120 mM NaCl, 5 mM KCl, 1 mM MgCl₂ and 2.5 mM CaCl₂ using an Ultra-Turrax homogenizer. The homogenate was centrifuged at 27,000×g for 10 min. The supernatant was discarded and the pellet is re-suspended in fresh buffer and centrifuged a second time. The final pellet was re-suspended in fresh buffer (35 ml per g of original tissue) and used for binding assays.

Assay

Aliquots of 500 μl homogenate were added to 25 μl of test solution and 25 μl of ³H-cytisine (1 nM, final concentration), mixed and incubated for 90 min at 0-4° C. Non-specific binding (5-10% of total binding) was determined using 100 μM (−)-nicotine.

Solutions of test compounds and ³H-cytisine were prepared 22× the desired final concentration. Compounds were dissolved in 100% DMSO (10 mM stock), diluted in 48% ethanol-water, and tested in triplicate in serial 1:3 or 1:10 dilutions. Reference compounds were not included routinely, but for every assay total and non-specific binding were compared to data obtained during validation of the assay.

Binding was terminated by rapid filtration onto Whatman GF/B glass fibre filters using a Brandel Cell Harvester, followed by seven washes with 2 ml ice-cold buffer. The amount of radioactivity on the filters was determined by conventional liquid scintillation counting using a Tri-Carb™ counter (PerkinElmer Life and Analytical Sciences).

Specific binding was calculated as total binding minus non-specific binding.

In Vitro Inhibition of ¹²⁵I-α-Bungarotoxin Binding (Rat Brain)

α-Bungarotoxin is a peptide isolated from the venom of the Elapidae snake Bungarus multicinctus and has high affinity for neuronal and neuromuscular nicotinic receptors, where it acts as a potent antagonist. ¹²⁵I-α-Bungarotoxin labels nAChRs formed by the α₇ subunit isoform found in brain and the α₁ isoform in the neuromuscular junction.

Tissue Preparation

Preparations were performed at 0-4° C. unless otherwise indicated. Cerebral cortices and hippocampi from male Wistar rats (150-250 g) were homogenized for 10 sec in 15 ml Tris, HCl (50 mM, pH 7.4) containing 120 mM NaCl, 5 mM KCl, 1 mM MgCl₂ and 2.5 mM CaCl₂, using an Ultra-Turrax homogenizer. The tissue suspension was centrifuged at 27,000×g for 10 min. The supernatant was discarded and the pellet was washed twice by centrifugation at 27,000×g for 10 min in 20 ml fresh buffer, and the final pellet was resuspended in fresh buffer containing 0.01% BSA (70 ml per g of original tissue) and used for binding assays.

Assay

Aliquots of 500 μl homogenate were added to 25 μl of test solution and 25 μl of ¹²⁵I-α-bungarotoxin (1 nM, final concentration), mixed and incubated for 2 h at 37° C. Non-specific binding was determined using (−)-nicotine (1 mM, final concentration). After incubation the samples were added 5 ml of ice-cold Tris buffer containing 0.05% PEI and poured directly onto Whatman GF/C glass fibre filters (pre-soaked in 0.1% PEI for at least ½ h) under suction and immediately washed with 2×5 ml ice-cold buffer. The amount of radioactivity on the filters was determined by conventional liquid scintillation counting. Specific binding was calculated as total binding minus non-specific binding.

In Vitro Inhibition of ¹²⁵I-α-bungarotoxin Binding to TE671 Cells

The neuromuscular nAChRs subtype—composed of α₁β₁γδ subunits—can be studied in the human medulloblastoma cell line TE671, and the α₁ subunit can be specifically labelled with ³H-α-bungarotoxin.

Tissue Preparation

TE671 cells were grown in Dulbecco's modified Eagle's medium, containing 10% horse serum and 5% fetal calf serum, in polystyrene culture flasks (175 cm²) in a humidified atmosphere of 5% CO₂ in air, at 37° C. Binding assays were conducted with cellular membrane fractions. Confluent TE671 cells were rinsed with 5 ml of PBS, and intact cells were harvested mechanically, i.e. by scraping the bottom of the culture flask with a rubber policeman after addition of 5 ml of PBS and then harvesting the dislodged cells by trituration. After determination of the number of recovered cells, the cell suspension was frozen at −80° C.

Assay

At the day of experiment, the cell suspension was thawed and centrifuged at 2° C. for 10 min (27.000×g), and the pellet was washed twice with 20 ml of ice-cold Tris, HCl (50 mM, pH 7.4) containing 120 mM NaCl, 5 mM KCl, 1 mM MgCl₂ and 2.5 mM CaCl₂. The final pellet was resuspended in Tris buffer containing 0.01% BSA (4×10⁶ cells/ml) and used for binding assays.

Aliquots of 0.5 ml membrane suspension were added to 0.025 ml of test solution and 0.025 ml of ¹²⁵I-α-bungarotoxin (1 nM, final concentration), mixed and incubated for 2 h at 37° C. Non-specific binding is determined using d-tubocurarine (0.1 mM, final concentration). After incubation the samples were poured directly onto Whatman GF/C glass fibre filters (pre-soaked in 0.1% PEI for at least 30 min) under suction and immediately washed with 2×5 ml ice-cold buffer. The amount of radioactivity on the filters was determined by conventional liquid scintillation counting. Specific binding is calculated as total binding minus non-specific binding.

Results

9-methyl-3-pyridin-3-yl-3,9-diaza-bicyclo[3.3.1]nonane -mediated in vitro inhibition of ³H-cytisine, ³H-epibatidine and ¹²⁵I-α-bungarotoxin binding were determined at rat brain tissue preparations and cell lines. Low nanomolar affinity for 9-methyl-3-pyridin-3-yl-3,9-diaza-bicyclo[3.3.1]nonane was observed at α4β2 (rat cortex ³H-cytisine binding) and α6/α3β2β3^{V273S}, whereas a lesser amount of affinity was detected for α7 (rat brain ¹²⁵I-α-bungarotoxin binding) and the neuromuscular α1-containing (TE671 ¹²⁵I-α-bungarotoxin binding) receptor subtypes. This demonstrates that 9-methyl-3-pyridin-3-yl-3,9-diaza-bicyclo[3.3.1]nonane bind with high affinity to α4β2 and α6-containing receptors, whereas lower affinity is observed at α7 and the neuromuscular α1-containing receptors.

TABLE 1
Affinities for 9-methyl-3-pyridin-3-yl-3,9-diaza-bicyclo[3.3.1]nonane.

| Ligand | Tissue/cell line | Kᵢ(nM) |
|---|---|---|
| ³H-epibatidine | α6/α3β2β3^{V273S} | 6.5 |
| ³H-cytisine | Rat cortex | 3.1 |
| ¹²⁵I-α-bungarotoxin | Rat brain | 290 |
| ¹²⁵I-α-bungarotoxin | TE671 | 320 |

### Example 4

### In Vivo Binding

In vivo binding studies have demonstrated that ³H-epibatidine binds with high-affinity to nicotinic receptors in the brain. Accumulation of ³H-epibatidine occurs preferentially in brain regions containing nicotinic receptors. The greatest concentration of radioactivity occurs in regions that are known to have high densities of nicotinic receptors i.e. thalamus and superior colliculus. The specific binding in thalamus reaches a maximum 30 min after an i.v. injection of ³H-epibatidine and this maximum is maintained for another 30 min. This specific binding of ³H-epibatidine can be partly or completely prevented by simultaneous or prior administration of drugs that to inhibit ligand binding to the receptors.

All test substances were administered as solutions or suspensions prepared in vehicle (e.g. saline, water, 5% glucose, 0.5% CMC, 0.5% HPMC or 10% HPβCD) and tested in serial 1:3 dilutions. Doses were adjusted for salt.

Groups of three female NMRI mice (25 g) were administered vehicle or test substance p.o. at a volume of 0.75 ml. Mice were injected i.v. via the tail vein with 1 μCi of ³H-epibatidine in 0.2 ml saline 45 min before decapitation. At the time of decapitation, the thalamus and a piece of cerebellum were rapidly dissected on ice. Tissues were weighed and dissolved for 36 h with 1 ml 2% sodium-laurylsulfate. The solubilized tissue was then added 2 ml of scintillation cocktail, and the amount of radioactivity in the tissue was counted by conventional liquid scintillation counting. Groups of vehicle-treated mice served as controls. Non-specific binding was defined as the amount of binding in cerebellum in vehicle treated mice.

Specific binding was determined as the amount of binding (dpm/5 mg tissue) in thalamus minus the amount of binding in cerebellum (dpm/5 mg tissue) in vehicle mice.

Results

The specific binding of ³H-epibatidine can be prevented by simultaneous or prior administration of drugs known to inhibit ligand binding to nicotinic receptors. In mice pre-dosed for 45 min with 9-methyl-3-pyridin-3-yl-3,9-diaza-bicyclo[3.3.1]nonane (p.o.) an ED₅₀ of 1.3 mg/kg was obtained.

In an in vivo time course study, mice were dosed with 2 mg/kg (p.o.) and inhibition of the specific binding of ³H-epibatidine was determined at time points up to 6 hours.

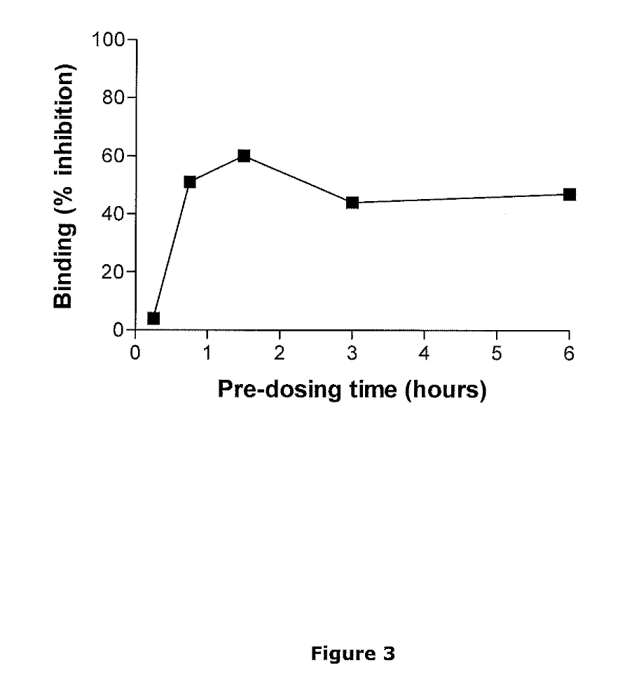

This study demonstrates that 9-methyl-3-pyridin-3-yl-3,9-diaza-bicyclo[3.3.1]nonane, following an initial period of low exposure, displays a maintained brain exposure for at least 6 hours (see FIG. 3), suggesting a long T½ which potentially could suggest a once-daily dosing scheme.

### Example 5

### Dopamine Release

Synaptosomal Preparation

Brains from Sprague-Dawley or Wistar rats (200-400 g) were dissected. Tissue from three rat brains yields enough material for one 96-well plate. Striata were dissected on an ice-chilled platform and placed in 12 ml ice-cold dissection buffer. The tissue was hereafter homogenized for 5-10 sec using a motor driven Teflon pestle in a glass homogenizing vessel. The homogenate was centrifuged at 1000×g for 10 min at 4° C. The resulting supernatant was then re-centrifuged at 12,000×g for 20 min at 4° C. The final crude P2 synatosomal fraction was re-suspended in oxygenated (equilibrated with an atmosphere of 96% O₂: 4% CO₂ for at least 30 min) Krebs bicarbonate buffer (0.5 ml/100 mg wet tissue weight) containing 100 nM ³H-dopamine (3.9 μl/100 mg wet tissue weight) and incubated at 37° C. for 10 min. Pargyline was added to the buffer to prevent degradation of ³H-dopamine.

Release Assay

The ³H-dopamine loaded synaptosomes were centrifuged at 1000×g for 5 min at room temperature. The pellet was re-suspended in 10 ml Krebs bicarbonate buffer containing 1 μM nomifensine (to inhibit re-uptake of ³H-DA during the experiment) and sedimented as described above. The washed synaptosomes were re-suspended in 11 ml Krebs bicarbonate buffer containing 1 μM nomifensine.

A 96-well Millipore filter plate (MSFBN6B50) was prewashed with 75 μl/well Krebs bicarbonate buffer (+ nomifensine) and the prewash buffer was removed by centrifugation for 1 min at 750 rpm into a 96-well waste plate. Aliquots of 75 μl synaptosomal suspension was pipetted into each well. The suspension in the synaptosomal preparation was hereafter removed by centrifugation for 1 min at 750 rpm into a 96-well waste plate. Synaptosomes were washed by adding 75 μl/well Krebs bicarbonate buffer (+ nomifensine) followed by centrifugation for 1 min at 750 rpm into a 96-well waste plate. Immediately hereafter, aliquots of 75 μl Krebs bicarbonate buffer (+ nomifensine) were added to each well and the plate is allowed to incubate for 2 min at room temperature. Incubation was terminated by centrifugation for 1 min at 750 rpm into a 96 well View plate (PerkinElmer) to collect basal release.

Following collection of the basal release 75 μl of Krebs bicarbonate buffer (+ nomifensine), containing nicotine and additional K⁺ (according to the plate layout), was added to each well and allowed to incubate for an additional 2 min at room temperature. Stimulated release was collected in a second plate by means of centrifugation as described above.

Following the collection of stimulated release, 75 μl Solvable™ was added to each well and allowed to incubate for at least 45 min to extract remaining ³H-DA from the sample. Tissue lysate samples were collected by centrifugation for 1 min at 750 rpm into a third plate. After addition of 150 μl of Microscint™ 40 scintillant to each well of the collecting plates containing basal, stimulated and tissue lysate, plates were sealed and shaken until the wells look clear. Radioactivity from each collection was determined by conventional liquid scintillation counting using a Packard Topcount™ counter.

Reagents
Dissection buffer (0.32 M sucrose, 5 mM HEPES, adjusted to pH 7.4 with NaOH)
Krebs bicarbonate buffer (113 mM NaCl, 3 mM KCl, 1.2 mM MgSO₄, 2.4 mM CaCl₂, 1.2 mM KH₂PO₄, 25 mM NaHCO₃, 10 mM glucose, 15 mM HEPES, 10 μM pargyline, adjusted to pH 7.4 with NaOH)

Results

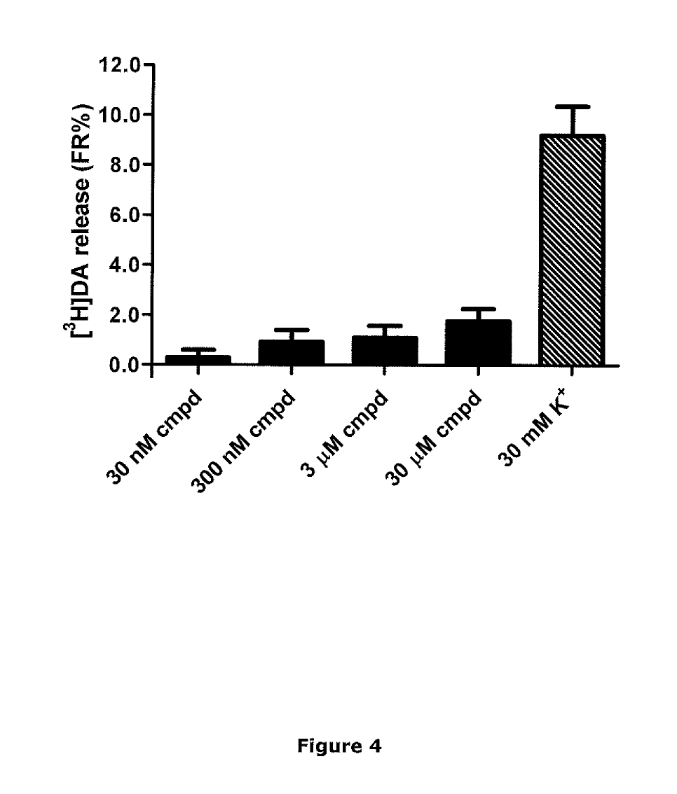

The graph in FIG. 4 illustrates the fraction of the total amount loaded ³H-dopamine which can be released upon stimulation. 9-Methyl-3-pyridin-3-yl-3,9-diaza-bicyclo[3.3.1]nonane concentration-dependently stimulates ³H-dopamine release from rat striatal synaptosomes. Release induced by a potassium-mediated depolarization (30 mM KCl) is depicted for comparison.

### Example 6

### Effect of 9-methyl-3-pyridin-3-yl-3,9-diaza-bicyclo[3.3.1]nonane on Reserpine-Induced Tremors in Rats

Parkinson's disease is associated with severe dopamine deficiency caused by neurodegeneration of dopaminergic cell bodies residing in Substantia Nigra pars compacta. As alpha6 nAChRs are enriched in the nigrostriatal dopamine pathway, activating this receptor may ameliorate symptoms in nigro-striatal dopamine deficiency models. Reserpine injections to rodents result in depletion of monoamines in the nigro-striatal dopamine pathway, resulting in catalepsy and tremors. Both behaviours can be quantified using dedicated tremor boxes. Pilot studies have shown that standard treatment for Parkinson's disease, L-DOPA (+ benserazide), as well as the dopamine D1/D2 agonist, apomorphine, reverse reserpine-induced tremors in rats.

Method

Six groups of male Sprague Dawley rats (250-300 grams) were subjected to the following treatment schedules, and effects on power spectra, assessed in automated tremor monitors were evaluated for 30 minutes:
Vehicle+vehicle
Vehicle+reserpine (1 mg/kg)
9-Methyl-3-pyridin-3-yl-3,9-diaza-bicyclo[3.3.1]nonane (0.1 mg/kg)+reserpine (1 mg/kg)
9-Methyl-3-pyridin-3-yl-3,9-diaza-bicyclo[3.3.1]nonane (1.0 mg/kg)+reserpine (1 mg/kg)
L-DOPA (100 mg/kg)+reserpine (1 mg/kg)
9-Methyl-3-pyridin-3-yl-3,9-diaza-bicyclo[3.3.1]nonane (1.0 mg/kg)+L-DOPA (100 mg/kg)+reserpine (1 mg/kg)

Reserpine was pre-treated i.v. 60 minutes prior to test start in a dose volume of 2 ml/kg. 9-Methyl-3-pyridin-3-yl-3,9-diaza-bicyclo[3.3.1]nonane was pre-treated s.c. 45 minutes prior to test start in a dose volume of 1 ml/kg. The dose of 9-methyl-3-pyridin-3-yl-3,9-diaza-bicyclo[3.3.1]nonane was based on 3H-epibatidin displacement studies showing half maximal displacement of specific binding of 3H-epibatidin in doses equaling 0.15 mg/kg in rats. L-DOPA was pre-treated i.p. 30 minutes prior to test start in a dose volume of 5 ml/kg. The dose was based on pilot studies demonstrating marginal activity per se of this dose, to see if 9-methyl-3-pyridin-3-yl-3,9-diaza-bicyclo[3.3.1]nonane was able to potentiate the effects of L-DOPA. All animals dosed with L-DOPA was also dosed with the peripherally acting decarboxylase inhibitor Benserazide to prevent the peripheral break down of L-DOPA (benserazide: 50 mg/kg, s.c., 30 min. prior to test start in a dose volume of 1 ml/kg).

Results

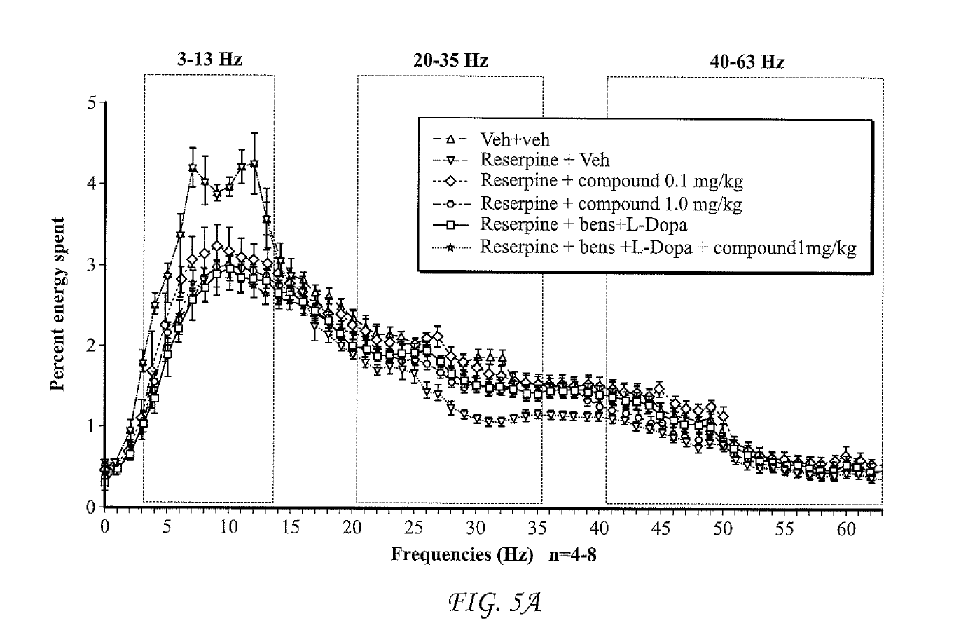

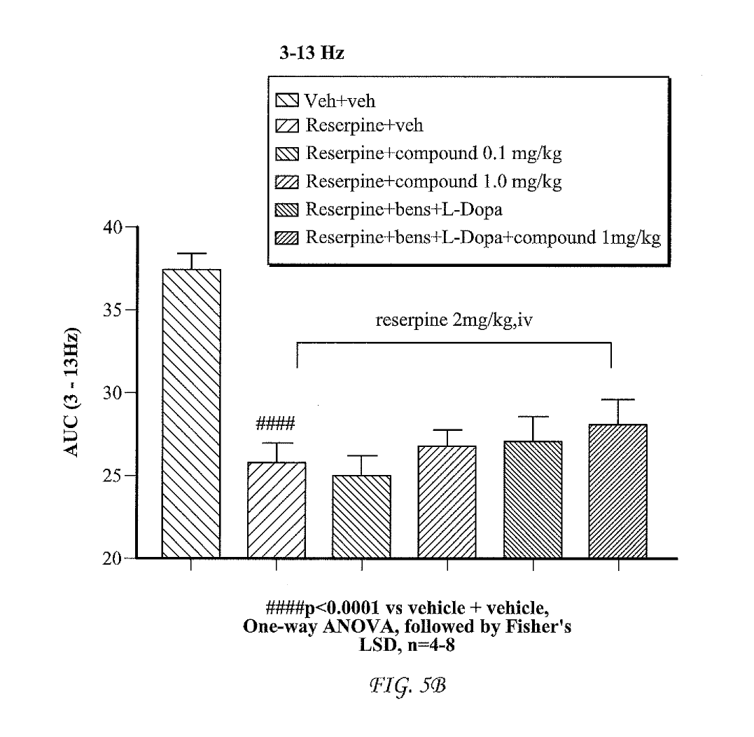

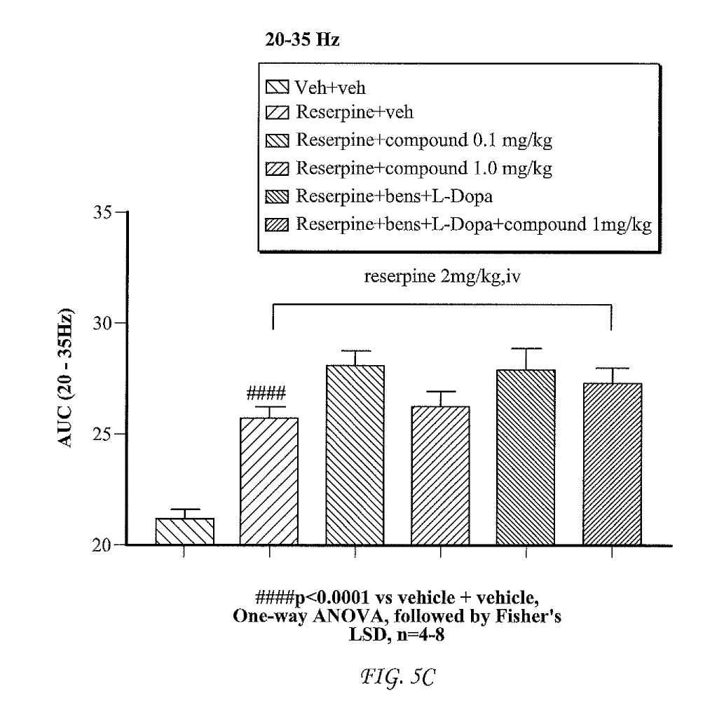

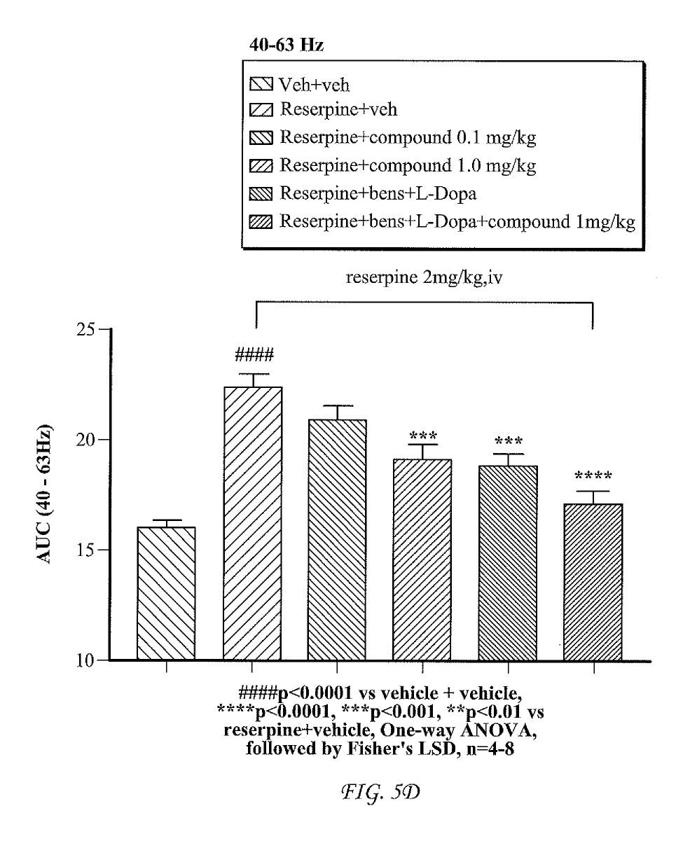

FIG. 5A represents the effects of the individual treatments on the full power spectra. Shaded areas depict the three different frequency ranges selected for calculation of Area Under the Curve (AUC) shown below (3-13 Hz, 20-43 Hz and 40-63 Hz).

Reserpine results in a marked reduction of low frequency movements (interpreted as catalepsy) as seen by significant reductions of movements in the 3-13 Hz range. Neither threshold dose of L-DOPA, nor 9-methyl-3-pyridin-3-yl-3,9-diaza-bicyclo[3.3.1]nonane or the combination of the two, was able to reverse this reduction.

In the higher frequency ranges, reserpine increases movements (interpreted as tremors) calculated as AUC for 20-43 Hz and 40-63 Hz, respectively. 9-Methyl-3-pyridin-3-yl-3,9-diaza-bicyclo[3.3.1]nonane results in a dose-related reversal of reserpine tremors reaching statistical significance at 1.0 mg/kg, in the high frequency range (40-63 Hz) specifically. Likewise, L-DOPA reduces reserpine-induced tremors in this frequency range specifically, without exerting any effects in the 20-43 Hz frequency range. Furthermore, when 9-methyl-3-pyridin-3-yl-3,9-diaza-bicyclo[3.3.1]nonane and L-DOPA were co-administered, there was a tendency for enhancement of the effects as compared to the individual treatments (FIG. 1D, 40-63 Hz). These data show that 9-methyl-3-pyridin-3-yl-3,9-diaza-bicyclo[3.3.1]nonane may be used to treat tremors associated with dopamine dysfunction.

### Example 7

### Effect of 9-methyl-3-pyridin-3-yl-3,9-diaza-bicyclo[3.3.1]nonane on MPTP-Induced Dopaminergic Neurotoxicity

The neurotoxicant 1-methyl-4-phenyl-1,2,3,6-tetrahydropyridine (MPTP) is a specific dopaminergic neuronal toxin that principally inhibits the multi-enzyme complex 1 of the mitochondrial electron transporter chain. MPTP is first converted to 1-methyl-4-phenyl pyridinium (MPP+) by astroglia and then enter neurons through DAT (dopamine transporter) causing specific dopaminergic neuronal death and leading to the clinical symptoms of Parkinson's disease in humans, primates and mice. For this reason, MPTP-induced dopaminergic neurotoxicity in mice is widely used as a model for Parkinson's disease research. It has been largely reported that MPP+ causes neurodegeneration of dopaminergic neuronal cultures and provides a useful model for Parkinson's disease in vitro.

Method

Pregnant female Wistar rats of 15 days gestation was euthanized by cervical dislocation and the foetuses was removed from the uterus. Hereafter, the embryonic midbrains was removed and placed in ice-cold medium of Leibovitz containing 2% of Penicillin-Streptomycin and 1% of bovine serum albumin (BSA). Only the ventral portions of the mesencephalic flexure were used for the cell preparations as this is the region of the developing brain rich in dopaminergic neurons. The midbrains were dissociated by trypsinisation for 20 min at 37° C. (Trypsin EDTA 1X). The reaction was stopped by the addition of DULBECCO′S MODIFIED Eagle's medium (DMEM) containing DNAse I grade II (0.1 mg/ml) and 10% foetal calf serum (FCS). Cells were then mechanically dissociated by 3 passages through a 10 ml pipette. Cells was then centrifuged at 180×g for 10 min at 4° C. on a layer of BSA (3.5%) in L15 medium. The supernatant was discarded, and the cells of pellet were re-suspended in a defined culture medium consisting of Neurobasal supplemented with B27 (2%), L-glutamine (2 mM) and 2% of PS solution and 10 ng/ml BDNF and 1 ng/ml of Glial cell-derived neurotrophic factor (GDNF). Viable cells were counted in a Neunauer cytometer using the tryphan blue exclusion test. The cells were seeded at a density of 40,000 cells/well in 96-well plates (wells were pre-coated with poly-L-lysine) and were hereafter cultured at 37° C. in a humidified air (95%)/CO2 (5%) atmosphere.

Half of the medium was changed every 2 days with fresh medium. In these conditions, after 5 days of culture, astrocytes are present in the culture and release growth factor allowing neuronal differentiation, and five to six percent of the neuronal cell populations were dopaminergic neurons. On day 6 of culture, the medium was removed and fresh medium with MPP+ (4 μM) was added (9-methyl-3-pyridin-3-yl-3,9-diaza-bicyclo[3.3.1]nonane at concentrations ranging from 1 nM to 1 μM was added 1H before intoxication). Following an additional 48H of incubation, the number of TH positive neurons were counted.

End Points Evaluation: Measure of Number of TH Positive Neurons

At the end of incubation time, cells were fixed by a solution of 4% paraformaldehyde for 20 min at room temperature. The cells were then permeabilized and non-specific sites were blocked with a solution of phosphate buffered saline (PBS) containing 0.1% saponin and 1% FCS for 15 min at room temperature. Cells were incubated with monoclonal Anti-Tyrosine Hydroxylase antibody produced in mouse, at a dilution of 1/10000 in PBS containing 1% FCS, 0.1% saponin, overnight at 4° C. Antibodies were revealed with Alexa Fluor 488 goat anti-mouse IgG in PBS with 1% FCS and 0.1% saponin for 1 h at room temperature. Nuclei of cells were labelled by a fluorescent marker (Hoechst solution) in the same solution.

For each condition, 20 pictures per well were taken using an InCell Analyzer™ 2000 with 20× magnification. Analysis of cell bodies of TH positive neurons was performed using Developer software (GE healthcare).

Results

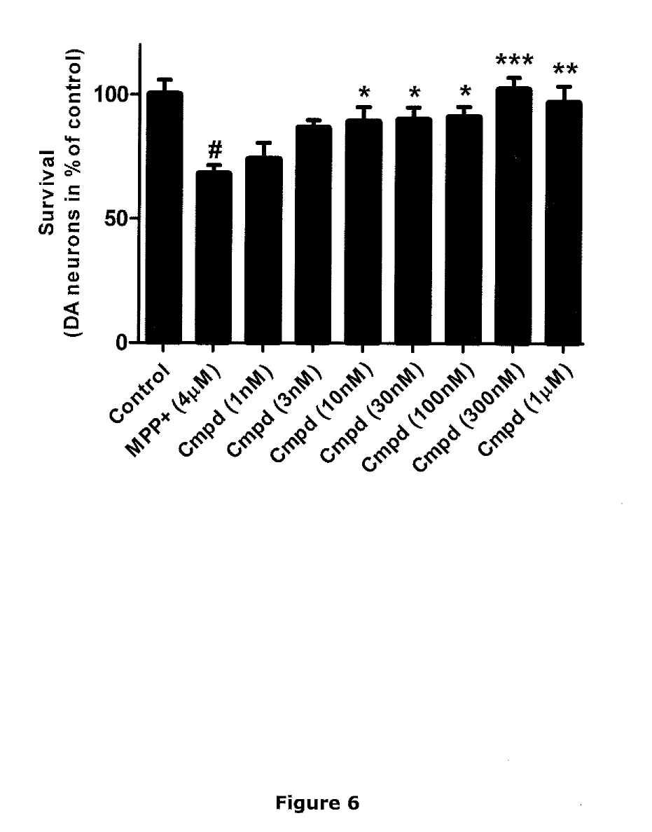

The neuroprotective effects of 9-methyl-3-pyridin-3-yl-3,9-diaza-bicyclo[3.3.1]nonane were evaluated in a primary dopaminergic neuronal culture injured by MPP+. Following exposure to MPP+ a general loss in the number of living dopaminergic neurons is observed. Co-treatment with 9-methyl-3-pyridin-3-yl-3,9-diaza-bicyclo[3.3.1]nonane demonstrates a concentration-dependent neuroprotective effect, with effective concentrations reflecting the potency measured at α6-containing nAChRs, see FIG. 6.

To conclude, this example demonstrates that 9-methyl-3-pyridin-3-yl-3,9-diaza-bicyclo[3.3.1]nonane is neuroprotective for dopaminergic neurons.

### Example 8

### Effects of 9-methyl-3-pyridin-3-yl-3,9-diaza-bicyclo[3.3.1]nonane on Dyskinesia in Parkinsonian Rats

This Example demonstrates the ability of systemic 9-methyl-3-pyridin-3-yl-3,9-diaza-bicyclo[3.3.1]nonane to alleviate L-dopa-induced dyskinesia in 6-OHDA lesioned rats.

Experimental Methods:

Subjects

Adult male Sprague-Dawley rats (Harlan labs), ˜3 months old and weighing 250-275 grams, were housed in groups of 2 in a temperature- and humidity-controlled colony room that was maintained on a 12 hour light/dark cycle. Food and water were available ad libitum throughout the experiment with the exception that animals were food fasted for 12 hours prior to the surgical (6-OHDA lesion) procedure.

Procedural Preparation

Prior to surgery, all animals were anesthetized and placed in the prone position. The hair was clipped from the head and the surgical site aseptically washed with betadine and alcohol. The animals head was fixed during surgery by a stereotaxic device and continuously anesthetized using isoflurane (1.5-2.0%) via a nosecone attached to the stereotaxic frame. The animal was draped with a sterile towel leaving only the surgical site exposed. The animals were monitored by the surgeon for suitable hemostasis and respiration.

Surgical Procedure

An incision was made extending through the skin and muscle to expose the skull. A surgical drill was used to create a small burr hole (1-1.5 mm diameter) over the cortex and striatum while leaving the dura intact. The dura was retracted exposing the cortical surface for injection the 6-OHDA. Two striatal sites (left striatum only) were injected with 10 μg 6-OHDA/site using a 28-gauge Hamilton syringe mounted to the stereotaxic frame at the following coordinates with respect to Bregma: (1) AP: 1.2; ML: 2.5, DV: −5.0 and (2) AP: 0.2; ML: 3.8, DV: −5.0. The 6-OHDA was infused in a volume of 2 μl per site over 2 minutes. The injection cannula was left in place for an additional 2 minutes allowing the 6-OHDA to diffuse from the injection site. After infusion, the skin was closed using Vicryl sutures.

Behavioral Testing

Treatments with L-dopa began 2 weeks after 6-OHDA lesions. To establish L-dopa abnormal involuntary movements (ATMs), rats received daily IP injections of L-DOPA (8 mg/kg; Sigma-Aldrich, Buchs, Switzerland) together with 15 mg/kg of benserazide (Sigma-Aldrich, Buchs, Switzerland) diluted in NaCl 0.9%, once a day for 3 weeks. A total of 12 rats received daily L-dopa. On day 21 of treatment, 8 rats were selected and matched for further testing based on a qualitative assessment of the severity and consistency of L-dopa-induced dyskinesia.

Animals were then tested to determine the extent of which 9-methyl-3-pyridin-3-yl-3,9-diaza-bicyclo[3.3.1]nonane attenuates L-dopa induced dyskinesia. Beginning on day 22 (post initiation of L-dopa) animals received saline or one of 3 doses of 9-methyl-3-pyridin-3-yl-3,9-diaza-bicyclo[3.3.1]nonane (Dose A=0.1 mg/kg sc, Dose B=0.3 mg/kg sc, or Dose C=1.0 mg/kg sc). 30 minutes prior to L-dopa. The number of animals used was minimized by using an experimental design in which each animal received each possible drug dose over time. Each treatment day was separated by 3-4 days according to the schedule listed below.

| | Test 1 | Test 2 | Test 3 | Test 4 |
|---|---|---|---|---|
| Animal/Test Day | Day 1 | Day 4 | Day 8 | Day 11 |
| Subject 1 | Saline | Dose A | Dose B | Dose C |
| Subject 2 | Dose A | Dose B | Dose C | Saline |
| Subject 3 | Dose B | Dose C | Saline | Dose A |
| Subject 7 | Dose C | Saline | Dose A | Dose B |
| Subject 8 | Saline | Dose A | Dose B | Dose C |
| Subject 10 | Dose A | Dose B | Dose C | Saline |
| Subject 11 | Dose B | Dose C | Saline | Dose A |
| Subject 12 | Dose C | Saline | Dose A | Dose B |

For quantification of L-DOPA-induced AIMs, rats were placed in transparent plastic cages and observed during the first minute of every 30-minute period in the 2 hours following the injection of L-DOPA. AIMs were classified into four subtypes as previously described [18]:

(1) axial AIMs, i.e., dystonic or choreiform torsion of the trunk and neck towards the side contralateral to the lesion;

(2) limb AIMs, i.e., jerky and/or dystonic movements of the forelimb contralateral to the lesion;

(3) orolingual AIMs, i.e., twitching of orofacial muscles, and bursts of empty masticatory movements with protrusion of the tongue towards the side contralateral to the lesion;

(4) locomotive AIMs, i.e., increased locomotion with contralateral side bias.

Each of the four subtypes was scored on a severity scale from 0 to 4.
0=absent
1=present during less than half of the observation time
2=present for more than half of the observation time
3=present all the time but suppressible by external stimuli
4=present all the time and not suppressible by external stimuli. Scores from these AIM subtypes were summed and used for statistical analyses.

At the conclusion of testing, we decided to test and additional higher dose of 9-methyl-3-pyridin-3-yl-3,9-diaza-bicyclo[3.3.1]nonane (3.0 mg/kg) using the testing schedule below.

| | Test 1 | Test 2 |
|---|---|---|
| Animal/Test Day | Day 13 | Day 15 |
| Subject 1 | Saline | 9-methyl-3-pyridin- |
| | | 3-yl-3,9-diaza- |
| | | bicyclo[3.3.1]nonane |
| | | (3.0 mg/kg) |
| Subject 2 | Saline | 9-methyl-3-pyridin- |
| | | 3-yl-3,9-diaza- |
| | | bicyclo[3.3.1]nonane |
| Subject 3 | Saline | 9-methyl-3-pyridin- |
| | | 3-yl-3,9-diaza- |
| | | bicyclo[3.3.1]nonane |
| Subject 7 | Saline | 9-methyl-3-pyridin- |
| | | 3-yl-3,9-diaza- |
| | | bicyclo[3.3.1]nonane |
| Subject 8 | 9-methyl-3-pyridin-3-yl- | Saline |
| | 3,9-diaza-bicyclo[3.3.1]nonane | |
| | (3.0 mg/kg) | |
| Subject 10 | 9-methyl-3-pyridin-3-yl- | Saline |
| | 3,9-diaza-bicyclo[3.3.1]nonane | |
| Subject 11 | 9-methyl-3-pyridin-3-yl- | Saline |
| | 3,9-diaza-bicyclo[3.3.1]nonane | |
| Subject 12 | 9-methyl-3-pyridin-3-yl- | Saline |
| | 3,9-diaza- bicyclo[3.3.1]nonane | |

Results

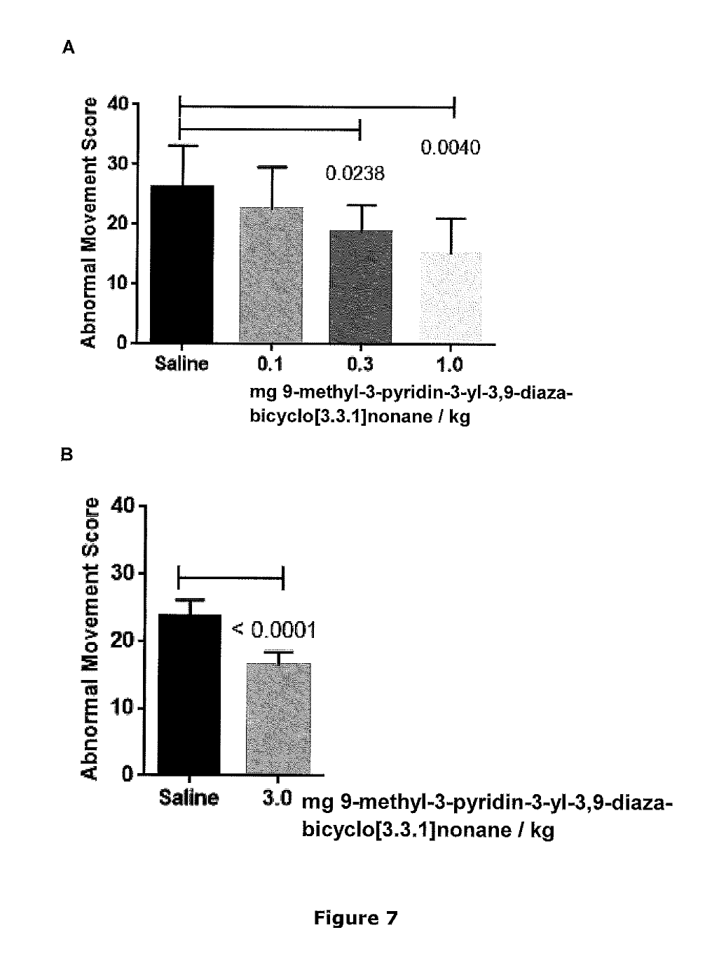

AIMs occurred in animals treated with daily L-dopa (8 mg) as previously described by Cenci et al. [18]. Qualitatively, the AIMs increased in frequency and severity between the first and second weeks of treatment with the axial and limb AIMs becoming most prominent. Administration of 9-methyl-3-pyridin-3-yl-3,9-diaza-bicyclo[3.3.1]nonane produced a significant, dose-related decrease in AIMs with the 0.3 and 1.0 mg/kg doses reaching statistical significance relative to saline controls (FIG. 7A). Treatment with a higher, follow-up dose of 3.0 mg/kg of 9-methyl-3-pyridin-3-yl-3,9-diaza-bicyclo[3.3.1]nonane did not further decrease L-dopa-induced AIMs (FIG. 7B).

To conclude, this Example demonstrate that 9-methyl-3-pyridin-3-yl-3,9-diaza-bicyclo[3.3.1]nonane is useful in alleviating L-dopa-induced dyskinesia.

### REFERENCES

[1] Koranda J L, Cone J J, McGehee D S, Roitman M F, Beeler J A, Zhuang X. Nicotinic receptors regulate the dynamic range of dopamine release in vivo. Journal of neurophysiology 2014; 111:103-11.

[2] Wonnacott S, Sidhpura N, Balfour D J. Nicotine: from molecular mechanisms to behaviour. Current opinion in pharmacology 2005; 5:53-9.

[3] Quik M, O′Neill M, Perez X A. Nicotine neuroprotection against nigrostriatal damage: importance of the animal model. Trends in pharmacological sciences 2007; 28:229-35.

[4] Perez X A, Quik M. Focus on alphα4betα2* and alpha6betα2* nAChRs for Parkinson's Disease Therapeutics. Molecular and cellular pharmacology 2011; 3:1-6.

[5] O′Neill M J, Murray T K, Lakics V, Visanji N P, Duty S. The role of neuronal nicotinic acetylcholine receptors in acute and chronic neurodegeneration. Current drug targets CNS and neurological disorders 2002; 1:399-411.

[6] Picciotto M R, Zoli M. Neuroprotection via nAChRs: the role of nAChRs in neurodegenerative disorders such as Alzheimer's and Parkinson's disease. Frontiers in bioscience: a journal and virtual library 2008; 13:492-504.

[7] Bordia T, Campos C, Huang L, Quik M. Continuous and intermittent nicotine treatment reduces L-3,4-dihydroxyphenylalanine (L-DOPA)-induced dyskinesias in a rat model of Parkinson's disease. The Journal of pharmacology and experimental therapeutics 2008; 327:239-47.

[8] Thacker E L, O′Reilly E J, Weisskopf M G, Chen H, Schwarzschild M A, McCullough M L, Calle E E, Thun M J, Ascherio A. Temporal relationship between cigarette smoking and risk of Parkinson disease. Neurology 2007; 68:764-8.

[9] Quik M, Perez X A, Grady S R. Role of alpha6 nicotinic receptors in CNS dopaminergic function: relevance to addiction and neurological disorders. Biochemical pharmacology 2011; 82:873-82.

[10] Gotti C, Moretti M, Bohr I, Ziabreva I, Vailati S, Longhi R, Riganti L, Gaimarri A, McKeith I G, Perry R H, et al. Selective nicotinic acetylcholine receptor subunit deficits identified in Alzheimer's disease, Parkinson's disease and dementia with Lewy bodies by immunoprecipitation. Neurobiology of disease 2006; 23:481-9.

[11] Quik M, Bordia T, Forno L, McIntosh J M. Loss of alpha-conotoxinMII- and A85380-sensitive nicotinic receptors in Parkinson's disease striatum. Journal of neurochemistry 2004; 88:668-79.

[12] Hone A J, Meyer E L, McIntyre M, McIntosh J M. Nicotinic acetylcholine receptors in dorsal root ganglion neurons include the α6β4* subtype. FASEB J. 2011; 26:917-926

[13] Liu L, Chang G Q, Jiao Y Q, Simon S A. Neuronal nicotinic acetylcholine receptors in rat trigeminal ganglia. Brain Res. 1998; 809:238-245

[14] Keiger C J, Walker J C. Individual variation in the expression profiles of nicotinic receptors in the olfactory bulb and trigeminal ganglion and identification of alpha2, alpha6, alpha9, and beta3 transcripts. Biochem Pharmacol. 2000; 59:233-240

[15] Genzen J R, Van Cleve W, McGehee D S. Dorsal root ganglion neurons express multiple nicotinic acetylcholine receptor subtypes. J Neurophysiol. 2001; 86:1773-1782

[16] Vincler M. Neuronal nicotinic receptors as targets for novel analgesics. Expert Opin Invest Drugs. 2005; 14:1191-1198

[17] Jeffrey S. Wieskopf, Jayanti Mathur, Walrati Limapichat, Michael R. Post, Mona A I-Qazzaz, et al. The nicotinic a6 subunit gene determines variability in chronic pain sensitivity via cross-inhibition of P2X2/3 receptors. Sci Transl Med. 2015; 7: pp. 287ra72

[18] Cenci M. A., Lindgren H S., Advances in understanding L-DOPA-induced dyskinesia, Curr Opin Neurobiol. 2007 December; 17(6):665-71. doi: 10.1016/j.conb.2008.01.004.

1. A method for treating, preventing, and/or alleviating a disease, disorder and/or condition that is a systemic atrophy primarily affecting the central nervous system, a Parkinsonian disorder, or pain in a subject comprising administering a therapeutically effective amount of 9-methyl-3-pyridin-3-yl-3,9-diaza-bicyclo[3.3.1]nonane, or a pharmaceutically acceptable salt thereof, to said subject.

2. The method according to claim 1, for the treatment, prevention, and/or alleviation of systemic atrophy primarily affecting the central nervous system.

3. The method according to claim 2, wherein the systemic atrophy primarily affecting the central nervous system is Huntington's disease.

4. The method according to claim 1, for the treatment, prevention, and/or alleviation of a Parkinsonian disorder.

5. The method according to claim 4, wherein the Parkinsonian disorder is Parkinson disease (PD), corticobasal degeneration (CBD), progressive supranuclear palsy (PSP), multiple system atrophy (MSA), dementia with Lewy bodies (DLB), Parkinson disease dementia, Levodopa-induced dyskinesia (LID), spinocerebellar atrophies (SCA), or frontotemporal dementia (FTD).

6. The method according to claim 5, wherein the Parkinsonian disorder is Parkinson disease.

7. The method according to claim 5, wherein the Parkinsonian disorder is Levodopa-induced dyskinesia.

8. The method of claim 5, further comprising administering L-DOPA, Benserazide, and/or Carbidopa to the subject.

9. The method according to claim 1, for the treatment, prevention, and/or alleviation of pain.

10. The method according to claim 9, wherein the pain is mild pain, moderate pain, severe pain, pain of acute character, pain of chronic character, pain of recurrent character, pain caused by migraine, postoperative pain, phantom limb pain, inflammatory pain, neuropathic pain, chronic headache, central pain, pain related to diabetic neuropathy, pain related to post therapeutic neuralgia, or pain related to peripheral nerve injury.

11. The method according to claim 1, wherein the 9-methyl-3-pyridin-3-yl-3,9-diaza-bicyclo[3.3.1]nonane is administered as a pharmaceutically acceptable salt.

12. The method according to claim 11, wherein the pharmaceutically acceptable salt of the 9-methyl-3-pyridin-3-yl-3,9-diaza-bicyclo[3.3.1]nonane is 9-methyl-3-pyridin-3-yl-3,9-diaza-bicyclo[3.3.1]nonane fumaric acid salt.

13. The method according to claim 1, wherein the 9-methyl-3-pyridin-3-yl-3,9-diaza-bicyclo[3.3.1]nonane, or pharmaceutically acceptable salt thereof, is administered in the form of a pharmaceutical composition comprising a therapeutically effective amount of the 9-methyl-3-pyridin-3-yl-3,9-diaza-bicyclo[3.3.1]nonane, or a pharmaceutically acceptable salt thereof, together with at least one pharmaceutically acceptable carrier, excipient, or diluent.

14. The method according to claim 13, wherein the pharmaceutical composition further comprises L-DOPA.

15. The method according to claim 13, wherein the pharmaceutical composition further comprises Benserazide and/or Carbidopa.

16. The method according to claim 1, wherein the 9-methyl-3-pyridin-3-yl-3,9-diaza-bicyclo[3.3.1]nonane or a pharmaceutically acceptable salt thereof is administered orally.

17. The method according to claim 1, wherein 0.1-500 mg of the 9-methyl-3-pyridin-3-yl-3,9-diaza-bicyclo[3.3.1]nonane or a pharmaceutically acceptable salt thereof is administered to said subject per day.

18. A kit comprising 9-methyl-3-pyridin-3-yl-3,9-diaza-bicyclo[3.3.1]nonane or a pharmaceutically acceptable salt thereof, and L-DOPA.

19. A composition comprising 9-methyl-3-pyridin-3-yl-3,9-diaza-bicyclo[3.3.1]nonane, or a pharmaceutically acceptable salt thereof, and L-DOPA.

20. The composition according to claim 19, further comprising Benserazide.

21. The composition according to claim 19, further comprising Carbidopa.

22. A method of
activating a nAChR, wherein the nAChR comprises at least one nAChRα₆,
comprising administering 9-methyl-3-pyridin-3-yl-3,9-diaza-bicyclo[3.3.1]nonane or a pharmaceutically acceptable salt thereof to the nAChR.

23. The method according to claim 22, wherein the 9-methyl-3-pyridin-3-yl-3,9-diaza-bicyclo[3.3.1]nonane acts as an agonist on the nAChRα₆.

24. The method according to claim 22, wherein the nAChR is expressed in a neuron.

25. The method according to claim 24, wherein the activation of the nAChR induces dopamine release from the neuron.

26. The method according to claim 24, wherein the activation of the nAChR stimulates neuronal survival.

27. The method according to claim 24, wherein the neuron is a neuron in substantia nigra pars compacta.

28. The method according to claim 24, wherein the neuron is a dopaminergic neuron.

29. The method according to claim 24, wherein the neuron is a tyrosine hydroxylase-positive neuron.
